(19) United States
(12) Patent Application Publication
(10) Pub. No.: US 2026/0259168 A1
(43) Pub. Date: Sep. 3, 2026

(54) FLEXIBLE MOLECULARLY IMPRINTED SENSOR FOR DETECTING ZEATIN RIBOSIDE, AND PREPARATION METHOD AND USE THEREOF

(71) Applicant: Intelligent Equipment Research Center, Beijing Academy of Agriculture and Forestry Services, Beijing (CN)

(72) Inventors: Aixue LI, Beijing (CN); Bin LUO, Beijing (CN); Le ZHANG, Beijing (CN); Wenxin YU, Beijing (CN); Dayu PAN, Beijing (CN); Peichen HOU, Beijing (CN); Tianyang LIU, Beijing (CN); Hongtu DONG, Beijing (CN)

(21) Appl. No.: 19/343,567

(22) Filed: Sep. 29, 2025

(30) Foreign Application Priority Data
Nov. 5, 2024 (CN) 202411568512.8

(51) Int. Cl. G01N 27/327; G01N 33/00; G01N 33/74

(52) U.S. Cl. CPC G01N 27/3275; G01N 33/0098; G01N 33/74; G01N 2333/415

(57) ABSTRACT

Disclosed are a flexible molecularly imprinted sensor for detecting zeatin riboside, a preparation method and use thereof. The preparation method includes preparing a laser-induced graphene pattern on a polyimide tape by a laser direct writing technology, and transferring the laser-induced graphene pattern onto polydimethylsiloxane, then modifying a composite material of zinc-aluminum layered double hydroxide and an iron-based metal-organic framework onto a working electrode, and then preparing a molecularly imprinted film on a surface of an electrode using o-phenylenediamine as a monomer and zeatin riboside as a template molecule, and eluting the template molecule to obtain the flexible molecularly imprinted sensor.

### CROSS REFERENCE TO RELATED APPLICATION

This patent application claims the benefit and priority of Chinese Patent Application No. 202411568512.8 filed with the China National Intellectual Property Administration on Nov. 5, 2024, the disclosure of which is incorporated by reference herein in its entirety as part of the present application.

### TECHNICAL FIELD

The present disclosure relates to the technical field of analysis and detection, and particularly relates to a flexible molecularly imprinted sensor for detecting zeatin riboside, a preparation method and use thereof.

### BACKGROUND

Cytokinin plays an important role in regulating plant growth, among which zeatin riboside (ZR) is a purine-type endogenous cytokinin in plants, widely present in higher plants, especially in actively growing parts such as root tips and leaves of plants. Its main functions include promoting cell division, regulating cell differentiation, delaying leaf senescence, and promoting lateral bud growth. In addition, it can help plants cope with abiotic stresses such as drought and light exposure by regulating stomatal aperture and density.

The ZR level in plants is generally low. Traditionally, methods used for ZR detection are mostly chromatographic methods and immunoassays. These methods have low detection efficiency, are mainly performed in vitro, and the detection equipment is costly and complex to operate. Compared with other methods, electrochemical sensors are lower in cost, simple to operate, portable, highly accurate in detection, and faster in response speed, and could be applied in the field of in-vivo detection of small molecules in plants.

However, plant tissues are very soft and mostly irregular in shape. Traditional sensors based on rigid electrodes (such as glassy carbon electrodes and gold electrodes) could not achieve effective fitting to a plant surface, which reduces the reliability and accuracy of the detection results. Therefore, how to design a flexible electrode capable of in-situ and in-vivo detection of zeatin riboside in plants has become a technical problem urgently needing to be solved in the field.

### SUMMARY

To solve the above technical problem, the present disclosure provides a method for preparing a flexible molecularly imprinted sensor for detecting zeatin riboside, including: modifying a surface of an laser-induced graphene/polydimethylsiloxane (LIG/PDMS) electrode with a zinc aluminum layered double hydroxide (ZnAl-LDH) and iron-based metal-organic frameworks (MIL-101 (Fe)) composite material to obtain a ZnAl-LDH-MIL-101 (Fe)/LIG/PDMS electrode, and preparing a molecularly imprinted film (MIP) on a surface of the ZnAl-LDH-MIL-101 (Fe)/LIG/PDMS electrode using o-phenylenediamine as a monomer and zeatin riboside as a template molecule, and then eluting the template molecule to obtain the flexible molecularly imprinted sensor.

ZnAl-LDH is a layered double hydroxide (LDHs) composed of zinc (Zn) and aluminum (Al), exhibiting good stability and catalytic properties. In the present disclosure, ZnAl-LDH is compounded with an iron-based metal-organic framework (MOF) material (MIL-101 (Fe)) to obtain a composite material, and the composite material is co-modified onto the surface of the flexible LIG/PDMS electrode, so that the LIG/PDMS electrode exhibits excellent stability and electroactivity when detecting zeatin riboside, and provides a stable response current for the sensor. By further combining with molecular imprinting technology, the flexible molecularly imprinted sensor exhibits an excellent detection effect during in-situ and in-vivo detection, achieving highly selective detection of ZR.

In some embodiments, the modifying the surface of the LIG/PDMS electrode with the ZnAl-LDH and the MIL-101 (Fe) composite material comprises: dissolving the ZnAl-LDH and the MIL-101 (Fe) in water and subjecting a resulting mixture to uniformly dispersing to obtain an ZnAl-LDH-MIL-101 (Fe) composite solution, and then dropping the ZnAl-LDH-MIL-101 (Fe) composite solution on a surface of a working electrode of the LIG/PDMS electrode to obtain the ZnAl-LDH-MIL-101 (Fe)/LIG/PDMS electrode.

In some embodiments, the ZnAl-LDH and MIL-101 (Fe) are dissolved in water at a mass ratio of 1:2 to 1:10, uniformly dispersed to obtain the ZnAl-LDH-MIL-101 (Fe) composite solution.

In some embodiments, preparing the molecularly imprinted film includes: mixing a zeatin riboside solution with an o-phenylenediamine solution to obtain a mixed solution; then placing the ZnAl-LDH-MIL-101 (Fe)/LIG/PDMS electrode into the mixed solution, performing electropolymerization by cyclic voltammetry, and washing and drying, and then eluting the template molecule to obtain the flexible molecularly imprinted sensor.

In some embodiments, a concentration of the zeatin riboside in the zeatin riboside solution is 1 in a range of mM to 5 mM; and a concentration of the o-phenylenediamine in the o-phenylenediamine solution is in a range of 1 mM to 10 mM.

In some embodiments, the electropolymerization is performed under the following conditions: a voltage of 0.2 V to 1.0 V, an electropolymerization cycle number of 10 cycles to 30 cycles, and a scan speed of 25 mV/s to 100 mV/s.

In some embodiments, an acetic acid solution is used to elute the template molecule. In some embodiments, a concentration of the acetic acid solution is 5% by volume to 15% by volume.

In some embodiments, the LIG/PDMS electrode is prepared by the following steps: preparing a laser-induced graphene on a polyimide tape (PI) by a laser direct writing technology, and transferring the laser-induced graphene onto polydimethylsiloxane (PDMS), coating a reference electrode with Ag/AgCl paste, and encapsulating wires and a passivation area to obtain the LIG/PDMS electrode.

The LIG/PDMS electrode is a laser-induced graphene (LIG) electrode with good flexibility and stretchability.

The LIG/PDMS electrode includes a working electrode, a counter electrode, and a reference electrode.

In some embodiments, the present disclosure provides a flexible molecularly imprinted sensor prepared by the method of any one of the above embodiments.

In some embodiments, the present disclosure provides use of the flexible molecularly imprinted sensor in in-situ and in-vivo detection of zeatin riboside in plants.

In a specific implementation process, the plant is any plant containing zeatin riboside, including but not limited to fruits, vegetables, flowers, and crops.

In some embodiments, the present disclosure provides a method for in-situ and in-vivo detection of the zeatin riboside in plants, including: punching a hole in a surface of the plants, attaching the flexible molecularly imprinted sensor to a hole-punched site, dropping a buffer solution at the hole-punched site, and then establishing connection to an electrochemical workstation, and detecting a concentration of the zeatin riboside by differential pulse voltammetry.

In some embodiments, the punched localization may be a stem, a leaf, or a fruit of the plant.

In some embodiments, the concentration of zeatin riboside could be obtained by establishing a standard curve.

In some embodiments, a relationship curve between current and zeatin riboside concentration could be established by using zeatin riboside-phosphate buffer solutions of different concentrations (pH=7.2 to 7.4), so as to obtain a standard curve.

In some embodiments, detection parameters of the differential pulse voltammetry include: a potential of −0.2 to 0.6 V, a pulse width of 0.02 s, an amplitude of 0.05 V, a pulse period of 1 s, and a sampling width of 0.02 s.

Compared with the conventional technology, some embodiments of the present disclosure have the following beneficial effects:

The present disclosure provides the flexible molecularly imprinted sensor for detecting zeatin riboside, and the flexible molecularly imprinted sensor can realize in-situ and in-vivo detection of zeatin riboside in plants, which avoids the problem of mechanical mismatch between a rigid sensor and a soft plant tissue in in-vivo plant detection. The flexible molecularly imprinted sensor according to the present disclosure could still maintain stable detection performance within a moderate deformation range, and is beneficial to adapting to an irregular surface of a plant tissue, does not cause substantial damage to a detection site, provides a low-cost and high-accuracy detection tool for in-vivo plant detection, and provides a basis for understanding a physiological state of the plant.

### BRIEF DESCRIPTION OF THE DRAWINGS

FIG. 1 shows a process flow diagram of Example 1 of the present disclosure; and

FIG. 2 shows a comparative diagram of the detection performance of three sensors: MIP/ZnAl-LDH-MIL-101 (Fe)/LIG/PDMS, MIP/MgAl-LDH-MIL-101 (Fe)/LIG/PDMS, and MIP/ZnAl-LDH-Cu-MOF/LIG/PDMS.

### DETAILED DESCRIPTION OF THE EMBODIMENTS

In order to make objects, technical solutions, and advantages of the present disclosure clearer, the technical solutions of the present disclosure will be described clearly and completely below. Apparently, embodiments described are some rather than all of the embodiments of the present disclosure. On the basis of the embodiments of the present disclosure, all other embodiments that can be obtained by those of ordinary skill in the art without creative efforts shall fall within the scope of the present disclosure.

In the examples provided in this specification, specific technologies or conditions that are not specified may be implemented according to the technologies or conditions described in the literature in the field, or according to product manuals. Reagents or instruments used therein for which no manufacturers are specified are all conventional products that can be obtained through regular commercial channels. ZnAl-LDH (catalog number 103063) and MIL-101 (Fe) (catalog number 103320) were purchased from Jiangsu XFNANO Materials Tech Co., Ltd, China.

Endpoints of ranges and any values disclosed in this specification are not limited to the precise ranges or values, and such ranges or values should be understood to include values close to the ranges or values. For numerical ranges, endpoints of the respective ranges, endpoints of the ranges and individual point values, as well as individual point values may be combined with each other to obtain one or more new numerical ranges, and these numerical ranges shall be considered as specifically disclosed herein.

### Example 1

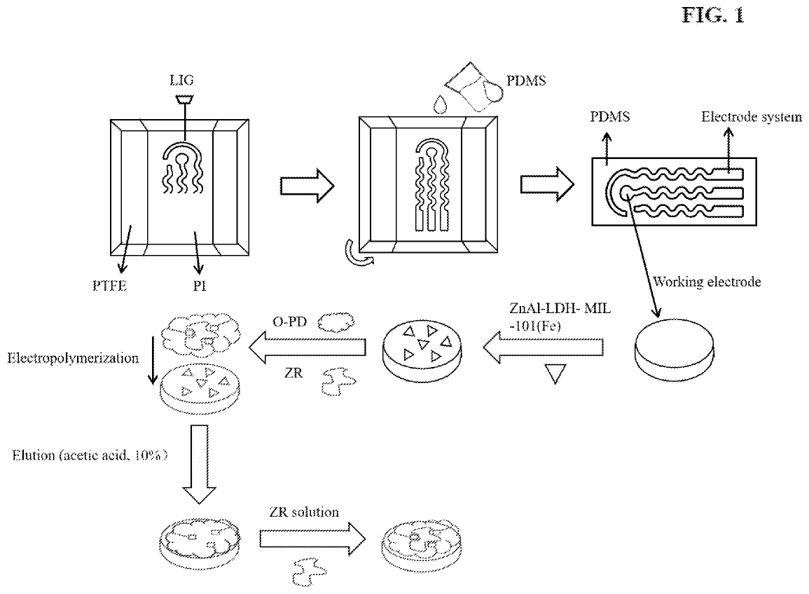

This example provided a flexible molecularly imprinted sensor for detecting zeatin riboside. A process flow diagram is shown in FIG. 1, and a preparation method thereof was conducted as follows:

(1) A polyimide (PI) tape was attached onto a 6×6 cm² polytetrafluoroethylene plate. An electrode pattern, consisting of a working electrode, a counter electrode, and a reference electrode, was designed using computer aided design (CAD). An exported electrode pattern was loaded into the software of a laser printing system with a printing power of 53%, and a printing depth of 10 μm to pattern a PI tape.

(2) A liquid polydimethylsiloxane (PDMS) was coated onto the polytetrafluoroethylene plate using a spin coater for 90 s, with a coating speed of 100 rpm. A resulting coated plate was then placed into a vacuum drying oven at 100° C. under vacuum for 12 h. Then, a PI film was peeled off from the PDMS to obtain a LIG/PDMS electrode.

(3) An appropriate amount of PDMS was applied to a non-working area of the LIG/PDMS electrode and cured by heating to avoid short circuits. Finally, Ag/AgCl paste was coated onto an exposed reference electrode and cured by heating.

(4) The LIG/PDMS electrode was placed in a phosphate buffer solution (0.1 mol/L, pH=7.2 to 7.4), and activated by a potentiostatic method (−1.7 V) for 180 s to remove impurities on a surface of the LIG/PDMS electrode.

(5) 1 mg of ZnAl-LDH and 5 mg of MIL-101 (Fe) were weighed and placed in 1 ml of ultrapure water. A resulting mixture was subjected to ultrasonication for 2 h to achieve uniform dispersion to obtain a ZnAl-LDH-MIL-101 (Fe) composite solution. 5 μL of the composite solution was dropped onto a surface of the working electrode and dried to obtain a ZnAl-LDH-MIL-101 (Fe)/LIG/PDMS.

(6) A 2 mM zeatin riboside solution was mixed with a 4 mM o-phenylenediamine solution in equal volume. The ZnAl-LDH-MIL-101 (Fe)/LIG/PDMS electrode was then placed in a resulting mixed solution. Electropolymerization was performed by cyclic voltammetry (CV) for 20 cycles, with a voltage range of −0.2 V to 1.0 V and a scan speed of 50 mV/s. After polymerization was completed, a residual polymerization solution was washed off with ultrapure water, and the ZnAl-LDH-MIL-101 (Fe)/LIG/PDMS electrode was dried. The ZnAl-LDH-MIL-101 (Fe)/LIG/PDMS electrode was then placed in a 10% acetic acid solution for 50 s to elute a template molecule, followed by washing and drying to obtain the flexible molecularly imprinted sensor for detecting zeatin riboside, MIP/ZnAl-LDH-MIL-101 (Fe)/LIG/PDMS.

### Example 2

In this example, the detection performance of the flexible molecularly imprinted sensor for detecting zeatin riboside prepared in an example was tested as follows:

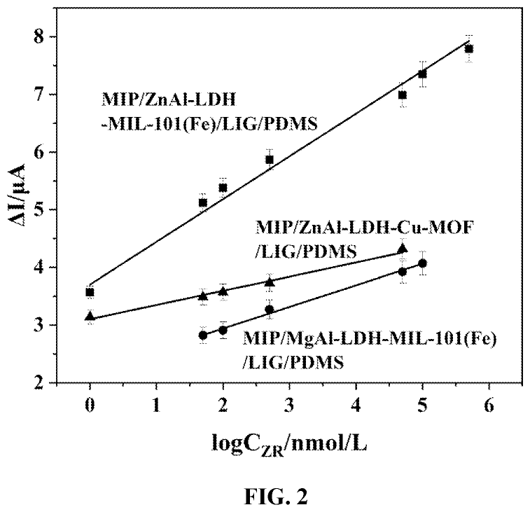

(1) Zeatin riboside-phosphate buffer solutions (pH=7.2 to 7.4) were respectively prepared at concentrations of 0, 1 nmol/L, 50 nmol/L, 100 nmol/L, 500 nmol/L, 50 μmol/L, 100 μmol/L, and 500 μmol/L. The flexible molecularly imprinted sensor prepared in Example 1 was connected to an electrochemical workstation (CHI 1040c). Detection was performed by differential pulse voltammetry (a potential of −0.2 to 0.6 V, a pulse width of 0.02 s, an amplitude of 0.05 V, a pulse period of 1 s, and a sampling width of 0.02 s). With the increase of a ZR solution concentration, an oxidation peak of Fc gradually decreased. A peak current of the oxidation peak obtained in a blank solution was recorded as I0, and the peak currents obtained in the standard zeatin riboside solutions of different concentrations were respectively recorded as I1, I2, I3, . . . By the formula AI=I0-I1 (I2, I3, . . . ), ΔI1, ΔI2, ΔI3, . . . were calculated respectively, thereby obtaining a set of relationship curves between the logarithm of ZR concentration and AI. A standard curve of zeatin riboside was prepared. As shown in FIG. 2, a linear detection range was 1 nmol/L to 500 μmol/L, and a detection limit was 0.387 nmol/L (S/N=3).

(2) Leaves of strawberry seedlings were ground and centrifuged. A supernatant was taken to detect the zeatin riboside level with reference to the standard curve. The supernatant was used as a matrix, and ZR standard samples (10 nmol/L, 50 nmol/L, 100 nmol/L) were gradually added, and a recovery rate was calculated. Moreover, liquid chromatography-mass spectrometry (LC-MS) was adopted for comparison. The chromatographic conditions were as follows: a chromatographic column was Eclipse XDB-C18 (250 mm×4.6 mm, 5 μm); a mobile phase was an aqueous solution of 0.1% formic acid+5 mmol/L ammonium acetate (A) and methanol (B), with a gradient elution program of 0~2.0 min. A proportion of phase A was maintained at 85% for 2.0 min to 5.0 min, the proportion of phase A decreased to 70% for 5.0 min to 7.0 min, the proportion of phase A decreased to 1% for 7.0 min to 8.0 min, the proportion of phase A was maintained at 1% with a flow rate of 0.3 mL/min, an injection volume of 1 μL, and a column temperature of 40° C. The mass spectrometry conditions were as follows: an agilent jet stream electrospray ionization (AJS EIS) source was used, and multiple reaction monitoring mode (MRM) detection was performed (positive ion scanning EMV +400 V, negative ion scanning EMV +500 V, with simultaneous positive and negative ion scanning), and a nebulizer gas pressure was 310.3 kPa (40 psi); a drying gas temperature and a flow rate were 300° C. and 12 L/min, respectively; and the capillary voltage was 3000 V for positive ions and 3500 V for negative ions.

The detection results are shown in Table 1.

TABLE 1
Detection of spiked recovery rate
of the sensor of Example 1 (n = 3)

| | Added | Detected | Recovery | |
|---|---|---|---|---|
| Juice concentration | concentration | concentration | rate | RSD |
| (nmol/L) | (nmol/L) | (nmol/L) | (%) | (%) |
| 24.5 (sensor), | 10 | 34.92 | 104.2 | 4.73 |
| 23.86 (LC-MS) | 50 | 74.31 | 99.62 | 5.81 |
| | 100 | 122.74 | 98.24 | 5.39 |

### Example 3

In this example, in-situ and in-vivo detection of zeatin riboside in strawberry leaves using the flexible molecularly imprinted sensor of Example 1 was conducted as follows:

Leaves of potted strawberry seedling were selected as experimental materials. A hole with a diameter of 1 mm was punched in a leaf using a hole punch. The flexible molecularly imprinted sensor of Example 1 was attached onto the leaf, and 20 μL of 10 mM phosphate buffer solution (PBS) was dropped at a hole-punched site. An electrochemical workstation (CHI 1040c) was connected, and a zeatin riboside concentration in the living strawberry leaf was calculated by differential pulse voltammetry (with testing conditions the same as in Example 2) in combination with the standard curve of Example 2. The results are shown in Table 2.

TABLE 2
Detection of ZR levels in strawberry leaves (nmol/L)

| Item | Sample 1 | Sample 2 | Sample 3 | Sample 4 | Sample 5 | Sample 6 | Mean value |
|---|---|---|---|---|---|---|---|
| Test | 14.84 | 18.72 | 15.94 | 13.46 | 11.97 | 14.45 | 14.897 ± |
| value | | | | | | | 2.304 |

### Comparative Example 1

This comparative example provided a flexible molecularly imprinted sensor for detecting zeatin riboside, a preparation method of which differs from that of Example 1 only in that:

ZnAl-LDH was replaced with an equal amount of MgAl-LDH to obtain a flexible molecularly imprinted sensor MIP/MgAl-LDH-MIL-101 (Fe)/LIG/PDMS.

A detection performance of the sensor was tested by the method in Example 2, and the results are shown in FIG. 2.

It can be seen that a linear detection range of the flexible molecularly imprinted sensor prepared in Comparative example 1 was 50 nmol/L to 100 μmol/L, with a detection limit of 2.17 nmol/L, and a detection effect was inferior to that of Example 1.

### Comparative Example 2

This comparative example provided a flexible molecularly imprinted sensor for detecting zeatin riboside, a preparation method of which differs from that of Example 1 only in that:

MIL-101 (Fe) was replaced with an equal amount of Cu-MOF, thereby obtaining a flexible molecularly imprinted sensor MIP/ZnAl-LDH-Cu-MOF/LIG/PDMS.

A detection performance of the sensor was tested by the method in Example 2, and the results are shown in FIG. 2.

It can be seen that a linear detection range of the flexible molecularly imprinted sensor prepared in Comparative example 2 was 1 nmol/L to 50 μmol/L, with a detection limit of 0.417 nmol/L, and a detection effect was inferior to that of Example 1.

It should be finally noted that the above embodiments are only intended to illustrate the technical solutions of the present disclosure but not to limit them. Although the present disclosure has been described in detail with reference to the foregoing embodiments, those of ordinary skill in the art should understand that they could still make modifications to the technical solutions described in the foregoing embodiments, or make equivalent substitutions for some of the technical features; and these modifications or substitutions do not cause the essence of the corresponding technical solutions to depart from the spirit and scope of the technical solutions of the embodiments of the present disclosure.

What is claimed is:

1. A method for preparing a flexible molecularly imprinted sensor for detecting zeatin riboside, comprising:
modifying a surface of a LIG/PDMS electrode with a ZnAl-LDH and MIL-101 (Fe) composite material to obtain a ZnAl-LDH-MIL-101 (Fe)/LIG/PDMS electrode, and
preparing a molecularly imprinted film on a surface of the ZnAl-LDH-MIL-101 (Fe)/LIG/PDMS electrode using o-phenylenediamine as a monomer and zeatin riboside as a template molecule, and then eluting the template molecule to obtain the flexible molecularly imprinted sensor.

2. The method of claim 1, wherein the modifying the surface of the LIG/PDMS electrode with the ZnAl-LDH and the MIL-101 (Fe) composite material comprises: dissolving the ZnAl-LDH and the MIL-101 (Fe) in water and subjecting a resulting mixture to uniformly dispersing to obtain a ZnAl-LDH-MIL-101 (Fe) composite solution, and then dropping the ZnAl-LDH-MIL-101 (Fe) composite solution on a surface of a working electrode of the LIG/PDMS electrode to obtain the ZnAl-LDH-MIL-101 (Fe)/LIG/PDMS electrode.

3. The method of claim 1, wherein the preparing the molecularly imprinted film comprises: mixing a zeatin riboside solution with an o-phenylenediamine solution to obtain a mixed solution; then placing the ZnAl-LDH-MIL-101 (Fe)/LIG/PDMS electrode into the mixed solution, performing electropolymerization by cyclic voltammetry, and washing and drying, and then eluting the template molecule to obtain the flexible molecularly imprinted sensor.

4. The method of claim 3, wherein a concentration of the zeatin riboside in the zeatin riboside solution is in a range of 1 millimole (mM) to 5 mM; and a concentration of the o-phenylenediamine in the o-phenylenediamine solution is in a range of 1 mM to 10 mM.

5. The method of claim 3, wherein the electropolymerization is performed under the following conditions: a voltage of 0.2 volt (V) to 1.0 V, an electropolymerization cycle number of 10 cycles to 30 cycles, and a scan speed of 25 millivolt per second (mV/s) to 100 mV/s.

6. The method of claim 3, wherein the eluting the template molecule is performed using a 5% to 15% acetic acid solution.

7. The method of claim 1, wherein the LIG/PDMS electrode is prepared by the following steps: preparing a laser-induced graphene on a polyimide tape by a laser direct writing technology, and transferring the laser-induced graphene onto polydimethylsiloxane, coating a reference electrode with Ag/AgCl paste, and encapsulating wires and a passivation area to obtain the LIG/PDMS electrode.

8. A flexible molecularly imprinted sensor for detecting zeatin riboside, which is prepared by the method of claim 1.

9. A method for in-situ and in-vivo detection of zeatin riboside in plants, comprising:
punching a hole in a surface of the plants, attaching the flexible molecularly imprinted sensor of claim 8 to a hole-punched site, dropping a buffer solution at the hole-punched site, and then establishing connection to an electrochemical workstation, and detecting a concentration of the zeatin riboside by differential pulse voltammetry.
